(19) United States
(12) Patent Application Publication
(10) Pub. No.: US 2011/0263697 A1
(43) Pub. Date: Oct. 27, 2011

(54) Methods of Reducing Oxidative Modification of a Muscle Cell Protein

(75) Inventor: Jeffrey J. Zachwieja, Cary, IL (US)

(73) Assignee: STOKELY-VAN CAMP, INC., Chicago, IL (US)

(21) Appl. No.: 13/092,199

(22) Filed: Apr. 22, 2011

Related U.S. Application Data
(60) Provisional application No. 61/327,324, filed on Apr. 23, 2010.

(51) Int. Cl. A61K 31/353; C12Q 1/02; C07D 311/72; A61P 39/06

(52) U.S. Cl. 514/456; 435/29; 549/405

(57) ABSTRACT

The present invention is directed to methods for reducing oxidative modification of one or more proteins or other cellular constituents involved in muscle cell contraction, for reducing muscle fatigue or for increasing muscle performance.

### RELATED APPLICATION

This application claims priority to U.S. Provisional Patent Application No. 61/327,324, filed on Apr. 23, 2010 and is hereby incorporated herein by reference in its entirety for all purposes.

### FIELD

The present invention relates to the field of improving muscle performance during exercise. The present invention also relates to the field of quenching reactive oxygen species in muscle tissue, thereby improving muscle performance. The present invention further relates to assays for determining the ability of a compound to improve muscle performance during exercise.

### BACKGROUND

Skeletal muscle cells produce oxidants as a result of strenuous contractions, such as during physical exercise. The oxidants, also known as reactive oxygen species, and/or free radicals, when produced in sufficient concentration in muscle cells during physical exercise, may affect cellular components of muscle cells that participate in the process of muscle cell contraction. See Matuszczak et al., Muscle & Nerve, 320:633-638 (2005); Clanton et al., Proc. Soc. Exp. Biol. Med. 1999; 222:253-262; Pattwell et al., Exerc. Sport Sci. Rev. 2004; 32:14-18; Reid, J. Appl. Physiol. 2001; 90:724-731; and Supinski, Mol. Cell Biochem. 1998; 179:99-110. See also, Powers et al., Physiol. Rev. 88:1243-1276 (2008), Zergeroglu et al., J. Appl. Physiol. 95:1116-1124 (2003), Betters et al., Am. J. Respir. Crit. Care Med. 170:1179-1184 (2004), and McArdle et al., Am. J. Physiol. 280:C621-C627 (2001) for additional studies on oxidative stress and muscle function.

Compounds having the capability of slowing or preventing the oxidation of other molecules are commonly referred to as “antioxidants.” Antioxidants are found in many fruits and vegetables and are widely used as ingredients in dietary supplements. Antioxidants are believed to quench reactive oxygen species and/or free radicals before they oxidize other molecules.

N-acetylcysteine (NAC), a reduced thiol donor believed to stimulate glutathione synthesis having antioxidant properties within a cell, has been shown to have an effect on muscle fatigue for certain moderate level muscle activity but not other intense, near-maximal muscle activity. See Matuszczak et al., Muscle & Nerve, 32:633-638 (2005). A whey protein based cysteine donor believed to augment intracellular GSH, which is believed to be an antioxidant, improved muscle performance in certain individuals. See Lands, J. Appl. Physiol 87:1381-1385 (1999). However, certain antioxidant supplements, like vitamin C, have been reported to appear to have no positive effects on muscle performance. See Matuszczak et al., Muscle & Nerve, 32:633-638 (2005); Powers et al., J. Sports Sci., 2004; 22:81-94.

It is therefore an object of the present invention to identify compounds, such as antioxidant supplements, that improve muscle cell performance. It is a further object of the present invention to create an assay effective to screen compounds for their ability to improve muscle cell performance. It is a still further object of the present invention to create methods of improving physical performance by administering a compound as a supplement that reduces oxidative modification of cellular components during physical exercise, thereby improving muscle performance by reducing muscle fatigue. These and other objects, features, and advantages of the invention or certain embodiments of the invention will be apparent to those skilled in the art from the following disclosure and description of exemplary embodiments.

### SUMMARY

Embodiments of the present invention are directed to compounds and methods for improving muscle cell performance during physical activity. Certain embodiments of the present invention are based on the discovery that compounds administered to a muscle cell reduce the oxidative modification of cellular proteins and other cellular components that occurs in a muscle cell as a result of contraction, such as elicited by electrical stimulation or during physical exercise by an individual. According to the present invention, muscle cells engaged in repeated contraction, such as by electrical stimulation or by physical exercise, create reactive oxygen species (ROS), oxidants and/or free radicals that, when present in a sufficient amount, oxidatively modify proteins involved in the muscle contraction process. This oxidative modification leads to muscle fatigue and reduced muscle cell performance. According to one aspect of the present invention, muscle cells will have increased ability, i.e. beyond a natural ability of the cell, to reduce oxidative modification of cellular proteins and other cellular components that otherwise occurs during physical exercise.

Compounds of the present invention exhibit antioxidant properties and are effective when administered directly to a muscle cell or to an individual as a supplement, food, meal replacement bar, confectioneries, snack foods or beverage product to quench reactive oxygen species, oxidants and/or free radicals that are created in the muscle cell as a result of contraction, such as by electrical stimulation or during physical exercise by the individual. The compounds increase the ability of the muscle cell to inhibit formation of oxidized cellular proteins and other cellular components. According to this aspect of the present invention, methods are provided to reduce muscle fatigue and/or loss of muscle performance by administering a compound that inhibits, reduces, limits and/or prevents oxidation of cellular proteins and cellular components associated with the process of muscle cell contraction. The compounds of the present invention supplement the inherent ability of a muscle cell to quench reactive oxygen species, oxidants and/or free radicals that are produced within the cell, such as during physical exercise. A reduction in the oxidative modification of cellular proteins and other cellular components beyond the muscle cell's own ability to do so, increases the ability of the muscle cell to continually contract before fatigue and/or muscle cell failure prevents or reduces further contraction.

Certain aspects of embodiments of the present invention include increasing the ability of a muscle cell to quench reactive oxygen species, oxidants and/or free radicals created in the muscle cell as a result of exercise. This aspect includes reducing the concentration of reactive oxygen species, oxidants and/or free radicals in the muscle cell as a result of exercise by administration of the compounds of the present invention, relative to a control muscle cell without administration of the compounds of the present invention. Aspects of the present invention include preventing, reducing or otherwise delaying the onset of muscle fatigue in an individual associated with exercise. Aspects of the present invention also include prolonging exercise time of an individual by reducing muscle fatigue. Still further aspects of the present invention include enhancing athletic performance in individuals by reducing muscle fatigue.

Embodiments of the present invention are also directed to a muscle cell assay and to methods of using a muscle cell assay to determine the ability of a compound to prevent, reduce, inhibit or limit oxidation of cellular proteins and other cellular components involved in the process of muscle contraction. The muscle cell assay of the present invention is used to quantify the amount of oxidative modification of contractile proteins and other cellular components involved in the process of muscle contraction. According to this aspect, muscle cells in a control assay are caused to contract by electrical stimulation for a period of time and at a frequency and pulse duration. Cellular proteins and other cellular components are analyzed to determine the extent of oxidative modification in the control relative to unstimulated or “resting” cells. Muscle cells are also contacted with a candidate compound and the muscle cells are caused to contract by electrical stimulation for the same period of time and at the same frequency and pulse duration. In a preferred embodiment, the candidate compound is an antioxidant. Cellular proteins and other cellular components are analyzed to determine the extent of oxidative modification which is then compared with the extent of oxidative modification in control and resting cells. If the extent of oxidative modification is reduced in the muscle cells contacted with the candidate compound, then the compound is selected and/or identified as a compound capable of reducing muscle fatigue and/or enhancing physical performance, for example in an individual.

According to an additional aspect, muscle cells in a control assay are caused to contract by electrical stimulation at a frequency and pulse duration and for a period of time until the point of failure, i.e., until the muscle cells stop contracting. Cellular proteins and other cellular components are analyzed to determine the extent of oxidative modification in the control. Muscle cells are also contacted with a candidate compound and the muscle cells are caused to contract by electrical stimulation at the same frequency and pulse duration until the point of failure. In a preferred embodiment, the candidate compound is an antioxidant. The time to failure for the control is compared with the time to failure for the candidate compound. If the time to failure is increased for the candidate compound, the candidate compound is selected and/or identified as a compound capable of reducing muscle fatigue and/or enhancing physical performance, for example in an individual.

In an additional aspect of the present invention, muscle cells are contacted with a control compound and are caused to contract by electrical stimulation at a specific pulse frequency and duration and for a period of time. According to a preferred embodiment, the control compound includes 6-hydroxy-2,5,7,8-tetramethylchroman-2-carboxylic acid which is a water soluble derivative of vitamin E commercially available under the name TROLOX and modifications and derivatives thereof to improve antioxidant capability. For example, various amino acids may be covalently attached to TROLOX to produce troloxylamino acids (T-AA) having greater antioxidant effectiveness that TROLOX. Examples include troloxyl-tryptophan-methyl ester and troloxyl-methionine-methyl ester as described in Taylor et al., Journal of the American Oil Chemists' Society, vol. 58, number 5, pp. 622-626 (1981) hereby incorporated by reference in its entirety for all purposes. Cellular proteins and other cellular components are analyzed to determine the extent of oxidative modification in the control. Muscle cells are also contacted with a candidate compound and the muscle cells are caused to contract by electrical stimulation for the same period of time and at the same pulse frequency and duration. In a preferred embodiment, the candidate compound is an antioxidant. Cellular proteins and other cellular components are analyzed to determine the extent of oxidative modification which is then compared with the extent of oxidative modification in the control. The ability of the candidate compound to reduce oxidation of cellular proteins and other cellular components is assessed as being higher or lower than the ability of the control compound to reduce oxidation of cellular proteins and other cellular components. In this manner, the capability of a compound to reduce muscle fatigue and/or enhancing physical performance, for example in an individual, is determined.

In a further aspect of the present invention, muscle cells are caused to contract by electrical stimulation to the point of fatigue and eventual failure. The time to failure is measured for the control, such as TROLOX. Muscle cells are also contacted with a candidate compound. In a preferred embodiment, the candidate compound is an antioxidant. The muscle cells are then caused to contract by electrical stimulation to the point of fatigue and eventual failure. The time to failure for the candidate compound is measured and compared to the time to failure for the control. If the time to failure for the candidate compound is longer than the time to failure for the control, then the candidate compound is selected as a compound capable of reducing muscle fatigue and/or enhancing physical performance, for example in an individual.

### BRIEF DESCRIPTION OF THE DRAWINGS

FIG. 1 depicts a flow chart showing the method steps of the OXYBLOT procedure used to visualize oxidatively modified proteins.

FIG. 2 depicts steps in the OXYBLOT procedure including SDS-gel electrophoresis and transfer, DNPH derivatization of oxidized protein and immunoblotting with primary and secondary antibody.

FIG. 3 is a graph depicting increased oxidative modification of myosin heavy chain protein in a muscle cell assay subjected to electrical pulse stimulation compared to a control with no electrical pulse stimulation.

FIG. 4 is a graph depicting reduced oxidative modification of myosin heavy chain protein in a muscle assay incubated with 1 mM TROLOX and subjected to electrical pulse stimulation compared to electrical pulse stimulation alone and a control with no electrical pulse stimulation.

### DETAILED DESCRIPTION OF CERTAIN EXEMPLARY EMBODIMENTS

Embodiments of the present invention are based on the discovery that proteins associated with the process of muscle cell contraction are oxidatively modified by reactive oxygen species, oxidants and/or free radicals generated in the muscle cell as a result of contractile activity, such as during exercise. The oxidative modification of proteins and other cellular constituents associated with the process of muscle contraction is a factor leading to muscle fatigue. According to aspects of the present invention, methods are provided to reduce oxidative stress in contracting skeletal muscle. In one embodiment, an antioxidant compound is administered to muscle cells in a manner to protect proteins and other cellular constituents associated with the process of muscle contraction from oxidative modification. According to this method of reducing oxidative modification, muscle fatigue is reduced and athletic performance is enhanced.

Proteins and other cellular constituents within the scope of the present invention that are involved in the process of muscle cell contraction and that are oxidized during muscle cell contraction include myosin heavy chain protein, Troponin C, actin, tropomyosin and regulatory proteins of the sarcoplasmic reticulum calcium release channels and the like and other components of the muscle contraction process described by Powers et al., Physiol Rev 88:1234-1276 (2008) hereby incorporated by reference in its entirety for all purposes.

According to an exemplary embodiment, myosin heavy chain protein is a protein involved in the process of muscle cell contraction. Myosin heavy chain protein is oxidized by reactive oxygen species, oxidants and/or free radicals that are produced during muscle cell contraction. This oxidation of myosin heavy chain protein results in a reduced ability of a muscle cell to contract, which is referred to herein as fatigue. Fatigue results in decreased athletic performance. Extensive oxidative modification of proteins and other cellular constituents involved in the muscle contraction process leads to muscle cell failure, i.e. failure to contract.

Compounds within the scope of the present invention that prevent, reduce, inhibit, limit and/or lower the oxidative modification of proteins and other cellular constituents, such as myosin heavy chain protein, involved in the muscle contraction process include compounds having antioxidant properties. Such compounds include naturally occurring antioxidant compounds and derivatives and modifications thereof. Such compounds also include synthetic antioxidants. A synthetic antioxidant is generally characterized as any compound having antioxidant capability and which is not a naturally occurring compound. Synthetic antioxidants include those created from organic synthesis.

Antioxidants within the scope of the present invention are compounds that are capable of slowing or preventing the oxidation of other molecules. Oxidation is a chemical reaction that transfers electrons from a substance to an oxidizing agent. When a compound is oxidatively modified or oxidized, its normal function can be inhibited. Oxidation reactions can produce free radicals, which start chain reactions that can compromise the function of proteins involved in the muscle contraction process. Antioxidants within the scope of the present invention terminate chain reactions generated by free radicals and inhibit other oxidation reactions by being oxidized themselves. It is to be understood that embodiments of the present invention include administration of one or more antioxidant compounds or a plurality of antioxidant compounds to reduce oxidative modification of cellular proteins and other cellular constituents involved in the muscle contraction process. Further, one or more antioxidant compounds or a plurality of antioxidant compounds are included in a liquid beverage or food product in amounts sufficient to reduce oxidative modification of cellular proteins and other cellular constituents involved in the muscle contraction process, when administered, delivered or otherwise ingested according to the methods described herein.

Antioxidants within the scope of the present invention include compounds having antioxidant properties selected from among polyphenols, tocopherols, tocotrienols, vitamins, vitamin cofactors, minerals, hormones, carotenoid terpenoids, flavonoid polyphenolics, phenolic acids and their esters, nonflavonoid phenolics and isomers, derivatives, glucose conjugates and modifications thereof. Common modifications include those to make an antioxidant compound more water soluble.

Exemplary vitamins within the scope of the present invention include vitamin A (retinol), vitamin C (ascorbic acid) and vitamin E including tocopherol and tocotrienol and derivatives and modifications thereof. One exemplary modification of vitamin E includes 6-hydroxy-2,5,7,8-tetramethylchroman-2-carboxylic acid which is a water soluble derivative of vitamin E commercially available under the name TROLOX.

Exemplary vitamin cofactors and minerals within the scope of the present invention include coenzyme Q10, manganese and iodide. Exemplary hormones within the scope of the present invention include melatonin. Exemplary carotenoid terpenoids within the scope of the present invention include alpha-carotene, astaxanthin, beta-carotene, canthaxanthin, lutein, lycopene, and zeaxanthin.

Exemplary flavonoid polyphenolics within the scope of the present invention include flavones such as apigenin, luteolin, tangeritin; flavonols such as isorhamnetin, kaempferol, myricetin, proanthocyanidins, quercetin and isoquercetin; flavanones such as eriodictyol, hesperetin, and naringenin; flavanols and their polymers such as catechin, gallocatechin and their corresponding gallate esters, epicatechin, epigallocatechin and their corresponding gallate esters, theaflavin and its gallate esters and thearubigins; isoflaflavone phytoestrogens such as daidzein, genistein and glycitein; stilbenoids such as resveratrol and pterostilbene; anthocyanins such as cyaniding, delphinidin, malvidin, pelargonidin, peonidin and petunidin; phenolic acids and their esters such as chicoric acid, chlorogenic acid, cinnamic acid, ferulic acid, ellagic acid, ellagitannins, gallic acid, gallotannins, rosmarinic acid, and salicylic acid.

According to aspects of the present invention, an antioxidant compound is administered to an individual for a time period before an exercise regimen, including a light exercise regimen, a moderate exercise regimen, a heavy exercise regimen or a competitive athletic event. Time periods within the scope of the present invention include about immediately before the exercise regimen or competitive athletic event, about 5 minutes to about 1 hour before the exercise regimen or competitive athletic event, about 1 to about 5 hours before the exercise regimen or competitive athletic event, about 1 to about 5 days before the exercise regimen or competitive athletic event and about 1 to about 5 weeks before the exercise regimen or competitive athletic event. The frequency of administration includes administration of the compound about every 30 minutes, about every hour, about every 2-3 hours, about every 3-5 hours, about every 12 hours, about every day, about every 3 days, about every week and the like. One of ordinary skill in the art will understand based on the benefit of this disclosure that the amount administered to an individual depends on at least the activity of the compound and its bioavailability in a particular individual. Certain amounts of a compound within the scope of the present invention include between about 1 milligram to about 10 grams, about 5 milligrams to about 5 grams, about 100 milligrams to about 3 grams, about 500 milligrams to about 2 grams and any and all ranges and values within the above ranges, whether overlapping or not. In a particular embodiment, about 1.25 grams of TROLOX are administered to an individual to achieve a blood concentration of about 1 mM. Further, certain amounts of a compound within the scope of the present invention include about 1 mg compound per kg of body weight (1 mg/kg) to about 100 mg/kg, about 5 mg/kg to about 50 mg/kg, about 10 mg/kg to about 25 mg/kg, about 15 mg/kg to about 20 mg/kg and any and all ranges and values within the above ranges, whether overlapping or not. According to certain aspect, such an amount is administered in a liquid volume of about 5 to about 30 fluid ounces, about 10 to about 25 fluid ounces, about 15 to about 20 fluid ounces and any and all ranges and values within the above ranges, whether overlapping or not. It is to be understood that frequency of administration may be dependent on the particular compound, the amount of compound or compounds administered, the mode of administration, the ability of the individual to metabolize the compound or compounds and the like. Further, the frequency and amount of compound or compounds administered may be determined by the desired amount of prevention, reduction, inhibition, limitation and/or lowering of the oxidative modification of proteins, such as myosin heavy chain protein, involved in the muscle contraction process. In general, larger amounts and more frequent administration may more strongly prevent, reduce, inhibit, limit and/or lower the oxidative modification of proteins. Still further, the frequency and amount of compound or compounds administered may also be determined by the desired amount of muscle fatigue reduction and/or improved muscle performance. Individuals desiring to reduce muscle fatigue or improve muscle performance to a greater extent may be more inclined to have greater frequency and higher amounts of compound or compounds administered.

According to aspects of the present invention, the compounds may be administered or delivered to an individual by ingestion by the individual such as by drinking a liquid such as a rehydration or sports beverage or enhanced water or shake, eating solid food products such as food bars, and ingesting eatable film strips, pills, lozenges, chews, gummies, gels, jellies, jellos, pastes, and the like. It is to be understood that the term administration is not limited to the providing of the compound by one individual to another. Instead, the term administration includes an individual providing the compound to herself or himself, such as by drinking a beverage including one or more antioxidant compounds.

One exemplary route of administration or delivery includes ingestion of the compound or compounds mixed in or otherwise dissolved or suspended in a fluid, such as with an emulsion. It is to be understood that aspects of the present invention include administration of one or more antioxidant compounds or a plurality of antioxidant compounds. Embodiments of the present invention also include fluids, drinks or beverages including one or more antioxidants in amounts sufficient to reduce oxidative modification of cellular proteins and other cellular constituents involved in the muscle contraction process, when administered, delivered or otherwise ingested according to the methods described herein. Fluids, drinks and/or beverages within the scope of the present invention include aqueous fluids, such as sports drinks or waters or enhanced waters that may further include one or more beverage ingredients to create a beverage, including carbonated or noncarbonated beverages. As an example, the antioxidant TROLOX, alone or in combination with other antioxidants, is included in a sports drink, enhanced water or other beverage in an amount sufficient to reduce oxidation of one or more proteins and/or other cellular components involved in muscle cell contraction that occurs during muscle contraction such as during exercise regimens or competitive athletic events or other strenuous physical activity, whether the individual is a trained athlete or not. In a particular embodiment, TROLOX is present in a sports drink or other beverage in an amount sufficient to reduce oxidative modification of myosin heavy chain protein so as to improve muscle and/or athletic performance.

It should be understood that liquids, sports drinks, rehydration beverages, beverages or other beverage products (all generally referred to as beverages or beverage products) in accordance with this disclosure may have any of numerous different specific formulations or constitutions. The formulation of a beverage product in accordance with this disclosure may vary to a certain extent, depending upon such factors as the product's intended market segment, its desired nutritional characteristics, flavor profile and the like.

Rehydration beverages including one or more antioxidants of the present invention in an amount sufficient to reduce oxidative modification of myosin heavy chain protein so as to improve muscle and/or athletic performance may be used in conjunction with physical activity, such as exercise, to replenish fluids and electrolytes lost during the activity as well as to provide additional energy. To this end, rehydration beverages typically comprise at least water, carbohydrates and electrolytes and have a measured osmolality of 250-350 mOsm/kg. The carbohydrates generally included in such beverages are high fructose corn syrup and sucrose. Other rehydration beverages include a carbohydrate blend that undergoes minimal hydrolysis in solution over time, thereby substantially maintaining its initial measured osmolality during storage. Further rehydration/sports beverages, containing such carbohydrate blends, have a low osmolality and are rapidly absorbed by a subject following consumption.

In accordance with one aspect, a carbohydrate blend is provided, which comprises from 35% by weight to 45% by weight fructose and from 55% by weight to 65% by weight glucose. The carbohydrate blend may include a combination of carbohydrates, such as fructose, glucose, sucrose, leucrose, trehalose, galactose, isomaltulose, dextrose, maltodextrin, corn syrup solids and/or glucooligosaccharides and combinations thereof. An aqueous solution containing 6% by weight of the carbohydrate blend has a measured osmolality of 230-300 mOsm/kg. Further, the measured osmolality of the 6% carbohydrate solution does not change by more than 5% during storage for up to six months.

In another aspect, a beverage composition is provided, comprising water and from 4% by weight to 10% by weight of a carbohydrate blend having from 35% by weight to 45% by weight fructose and from 55% by weight to 65% by weight glucose. The beverage may be a rehydration beverage and further include electrolytes, edible acids, vitamins, functional ingredients, coloring agents, flavoring agents and combinations thereof.

In certain embodiments of the carbohydrate blend and beverage composition disclosed here, at least some of the glucose is provided by glucooligosaccharides, which may have a structure containing between about three and seven degrees of saccharide polymerization or up to six degrees of saccharide polymerization, while in other embodiments the structure contains up to ten degrees of saccharide polymerization. In certain embodiments, at least some of the glucose is provided by polysaccharides having a degree of polymerization of eleven degrees and greater. In certain exemplary embodiments of beverage compositions according to this disclosure, a rehydration beverage is provided having a measured osmolality in the range of 230 mOsm/kg to 260 mOsm/kg. In certain embodiments of the present invention, the structure of the glucooligosaccharides included in the carbohydrate blend has an initial α-(1,4) glucose-to-glucose linkage followed by alternating α-(1,3) glucose-to-glucose linkages and α-(1,6) glucose-to-glucose linkages. A suitable glucooligosaccharide is produced by Cargill, Incorporated, Wyzata, Minn., under the name Glucohydrate.

Osmolality is defined as the number of osmoles of solute per kilogram of solvent, where one osmole is provided by each mole of ion charge. Glucooligosaccharides have larger molecular weights than smaller carbohydrates, such as disaccharides or monosaccharides. Accordingly, a first solution of a carbohydrate blend comprising a particular weight percent of glucooligosaccharides would have a lower osmolality than a second carbohydrate solution that is identical except that it instead comprises that particular weight percent of disaccharides in place of the glucooligosaccharides. The reason for this is because fewer total moles of carbohydrate would be present in the first solution than in the second solution. Consequently, whereas rehydration beverages comprising sucrose and high fructose corn syrup (HFCS) typically have an initial measured osmolality of about 330 mOsm/kg, an aqueous solution containing between about 4% by weight and about 10% by weight of the carbohydrate blend has a measured osmolality of about 230-300 mOsm/kg. Further, in certain exemplary embodiments of beverage compositions according to this disclosure, a rehydration beverage composition is provided having a measured osmolality in the range of 230 mOsm/kg to 260 mOsm/kg.

Beverage compositions according to different embodiments may comprise one or more carbohydrate source(s). In certain embodiments, the carbohydrates may include sources of monosaccharides, disaccharides and glucooligosaccharides, while in other embodiments the carbohydrates also include sources of polysaccharides, for example corn syrup solids. In certain embodiments, a beverage composition is provided that comprises water and from 4% by weight to 10% by weight of a carbohydrate blend having from 35% by weight to 45% by weight fructose and from 55% by weight to 65% by weight glucose. At least some of the glucose is provided by glucooligosaccharides. The beverage may be a rehydration beverage and further include electrolytes, edible acids, coloring agents, flavoring agents, vitamins, functional ingredients and combinations thereof.

Advantageously, certain embodiments of the present invention provide compositions, such as rehydration beverage compositions, in which the hydrolysis of the carbohydrate source is minimized. Because hydrolysis of carbohydrates results in an increase of the total number of moles of carbohydrate, the osmolality of compositions that are subjected to hydrolysis will exhibit an increase in measured osmolality over time. In contrast, the measured osmolality of compositions comprising 4% by weight to 10% by weight of a carbohydrate blend according to the present invention does not increase by more than 5% during storage for up to six months. Accordingly, compositions according to embodiments of the invention generally provide rehydration beverages that have a measured osmolality below that of plasma, (e.g., approximately 300 mOsm/kg), and are quickly absorbed by the gastrointestinal system both immediately, and for up to at least six months, following manufacture.

It will generally be an option to add further ingredients to the formulation of a particular beverage embodiment, including sports drinks or rehydration beverages. One or more sweeteners, flavorings, electrolytes, vitamins, fruit juices or other fruit products, tastents, masking agents and the like, flavor enhancers, and/or carbonation typically may be added to any such formulations to vary the taste, mouthfeel, nutritional characteristics, etc. In general, a beverage in accordance with this disclosure typically comprises at least water, one or more antioxidant compounds in accordance with the present invention, sweetener, acidulant, colorant and/or flavoring It is to be understood that beverage ingredients include both natural and artificial ingredients. Exemplary flavorings which may be suitable for at least certain formulations in accordance with this disclosure include fruit flavoring, cola flavoring, citrus flavoring, spice flavorings and others. Carbonation in the form of carbon dioxide may be added for effervescence. Preservatives may be added if desired, depending upon the other ingredients, production technique, desired shelf life, etc. Optionally, caffeine may be added. Certain exemplary embodiments of the beverages disclosed here are cola-flavored carbonated beverages, characteristically containing carbonated water, sweetener, kola nut extract and/or other flavoring, caramel coloring, phosphoric acid, and optionally other ingredients. Additional and alternative suitable ingredients will be recognized by those skilled in the art given the benefit of this disclosure.

The beverage products disclosed here include beverages, i.e., ready-to-drink liquid formulations, beverage concentrates and the like. As used herein, the term “ready-to-drink” refers to a beverage that can be ingested as-is. That is, the ready-to-drink beverage requires no dilution or additions prior to ingestion by a consumer. Beverage products include, e.g., sports drinks, carbonated and non-carbonated soft drinks, fountain beverages, frozen ready-to-drink beverages, coffee beverages, tea beverages, dairy beverages, powdered soft drinks, as well as liquid concentrates, flavored waters, enhanced waters, fruit juice and fruit juice-flavored drinks, and sport drinks. In a basic form, beverage ingredients include one or more of water, one or more antioxidant compounds, an edible acid, a flavorant, salts, sweeteners, a colorant, a preservative and mixtures of any of them. In embodiments providing a packaged ready-to-drink beverage, the beverage composition may be pre-mixed with a liquid such as water. In certain embodiments, the ready-to-drink beverage comprises about 80-99 weight percent (wt %) of liquid of the total weight of the beverage. Unless otherwise specified, all weight percentages are based on the total weight of a ready-to-drink beverage. In further embodiments, the beverage composition can be packaged as an edible composition or concentrate, such as a dry mix (e.g., powder) or a liquid concentrate for later reconstitution with one or more liquids to form a beverage. The concentrated composition may be associated with instructions for preparing the beverage composition. In another embodiment, a beverage concentrate may be packaged as gels, capsules, or tablets which are consumed with liquid. When provided in these forms, the beverage composition may comprise instructions to mix or consume with an amount of liquid which is equal to about 80-99 wt % of the prepared beverage composition.

The terms “beverage concentrate,” “throw beverage syrup” and “syrup” are used interchangeably throughout this disclosure. As used here “sweetened syrup” is defined as syrup that possesses sweetness, and comprises at least one or more sweeteners. At least certain exemplary embodiments of the beverage concentrates contemplated are prepared with an initial volume of water to which the additional ingredients are added. A single strength beverage composition (i.e., a beverage composition at a concentration that is ready to drink) may be formed from the beverage concentrate or syrup by adding further volumes of water to the concentrate to dilute it to a single strength. Typically, for example, single strength beverages may be prepared from the concentrates by combining approximately 1 part concentrate with between approximately 3 to approximately 7 parts water. In certain exemplary embodiments the single strength beverage is prepared by combining 1 part concentrate with 5 parts water. In certain exemplary embodiments the additional water used to form the single strength beverages is carbonated water. In certain other embodiments, a single strength beverage is directly prepared without the formation of a concentrate and subsequent dilution.

Natural embodiments of the beverage products disclosed here are natural in that they do not contain anything artificial or synthetic (including any color additives regardless of source) that would not normally be expected to be in the food. As used herein, therefore, a “natural” beverage composition is defined in accordance with the following guidelines: Raw materials for a natural ingredient exists or originates in nature. Biological synthesis involving fermentation and enzymes can be employed, but synthesis with chemical reagents is not utilized. Artificial colors, preservatives, and flavors are not considered natural ingredients. Ingredients may be processed or purified through certain specified techniques including at least: physical processes, fermentation, and enzymolysis. Appropriate processes and purification techniques include at least: absorption, adsorption, agglomeration, centrifugation, chopping, cooking (baking, frying, boiling, roasting), cooling, cutting, chromatography, coating, crystallization, digestion, drying (spray, freeze drying, vacuum), evaporation, distillation, electrophoresis, emulsification, encapsulation, extraction, extrusion, filtration, fermentation, grinding, infusion, maceration, microbiological (rennet, enzymes), mixing, peeling, percolation, refrigeration/freezing, squeezing, steeping, washing, heating, mixing, ion exchange, lyophilization, osmose, precipitation, salting out, sublimation, ultrasonic treatment, concentration, flocculation, homogenization, reconstitution, enzymolysis (using enzymes found in nature). Processing aids (currently defined as substances used as manufacturing aids to enhance the appeal or utility of a food component, including clarifying agents, catalysts, flocculants, filter aids, and crystallization inhibitors, etc. See 21 CFR §170.3(o)(24)) are considered incidental additives and may be used if removed appropriately. Substantially clear embodiments of the beverage products disclosed here are substantially clear in that the beverages have substantially no turbidity and substantially no color.

Water is a basic ingredient in the beverage products disclosed here, typically being the vehicle or primary liquid portion in which the antioxidant compound is provided and the remaining ingredients are dissolved, emulsified, suspended or dispersed. Purified water can be used in the manufacture of certain embodiments of the beverages disclosed here, and water of a standard beverage quality can be employed in order not to adversely affect beverage taste, odor, or appearance. The water typically will be clear, colorless, free from objectionable minerals, tastes and odors, free from organic matter, low in alkalinity and of acceptable microbiological quality based on industry and government standards applicable at the time of producing the beverage. In certain typical embodiments, water is present at a level of from about 80% to about 99.9% by weight of the beverage. In at least certain exemplary embodiments the water used in beverages and concentrates disclosed here is “treated water,” which refers to water that has been treated to reduce the total dissolved solids of the water prior to optional supplementation, e.g., with calcium as disclosed in U.S. Pat. No. 7,052,725. Methods of producing treated water are known to those of ordinary skill in the art and include deionization, distillation, filtration and reverse osmosis (“r-o”), among others. The terms “treated water,” “purified water,” “demineralized water,” “distilled water,” and “r-o water” are understood to be generally synonymous in this discussion, referring to water from which substantially all mineral content has been removed, typically containing no more than about 500 ppm total dissolved solids, e.g. 250 ppm total dissolved solids.

In one embodiment, the beverage composition includes an electrolyte source for providing sodium (Na). Sodium may be provided by compounds of sodium, such as sodium chloride, sodium citrate, sodium carbonate, sodium bicarbonate, sodium lactate, trisodium citrate, sodium gluconate, monosodium phosphate, disodium phosphate, trisodium phosphate, tetrasodium acid pyrophosphate, sodium acid sulfate, or combinations thereof. In one embodiment, the sodium is provided by sodium lactate, which is about 20.5% by weight sodium. In another embodiment, the sodium is provided by sodium chloride, which is about 39.4% by weight sodium. In a further embodiment, the sodium is provided by sodium acid sulfate, which is about 19.2% by weight sodium. In yet another embodiment, the sodium is provided by sodium gluconate, which is about 10.5% by weight sodium.

In select beverage embodiments, the amount of sodium is about 0.03% by weight to about 0.06% by weight of the finished product or combinations thereof. In select embodiments, the amount of sodium is about 0.03% by weight to about 0.06% by weight of the beverage. Other amounts may also be useful, depending on the application and other factors. In one embodiment, the sodium is provided by sodium chloride and sodium citrate.

Additional types of electrolyte sources to provide, for example, potassium (K), magnesium (Mg), calcium (Ca) and chloride (Cl) ions can also be included in the beverage composition in addition to or independently of sodium (Na). The different types of electrolytes can be provided by their compounds or a combination of their compounds.

For example, an electrolyte source for providing calcium includes potassium acetate, potassium bicarbonate, potassium bromide, potassium chloride, potassium citrate, potassium-D-gluconate, potassium phosphate such as mono- and dibasic potassium phosphate, tropotassium phosphate, tetrapotassium pyrophosphate, potassium sulfate, potassium acetate, potassium bicarbonate, potassium bromide, tripotassium citrate, calcium acetate, calcium chloride, calcium citrate, calcium-D-gluconate, calcium lactate, calcium laevulinate, dibasic calcium phosphate, magnesium chloride, magnesium carbonate and magnesium sulphate, or a combination thereof.

In certain embodiments, the electrolyte blend includes an electrolyte source for providing chloride (Cl). Chloride may be provided by compounds of chloride, such as magnesium chloride hexahydrate, potassium chloride, sodium chloride, anhydrous calcium chloride, or combinations thereof. In one embodiment, the chloride is provided by potassium chloride, which is about 47.5% by weight chloride. In another embodiment, the chloride is provided by sodium chloride, which is about 60.6% by weight chloride. In a further embodiment, the chloride is provided by calcium chloride (anhydrous), which is about 63.9% by weight chloride. In select beverage embodiments, the amount of chloride is about 0.03% by weight to about 0.06% by weight of the finished product.

In certain embodiments, the electrolyte blend includes an electrolyte source for providing calcium (Ca). Calcium may be provided by compounds of calcium, such as anhydrous calcium chloride, calcium acetate, calcium chloride, calcium citrate, calcium-D-gluconate, calcium lactate, calcium laevulinate, dibasic calcium phosphate. In one embodiment, the calcium is provided by calcium chloride anhydrous, which is about 36.1% by weight calcium. In select beverage embodiments, the amount of calcium is about 0.01% by weight to about 0.03% by weight of the finished product.

Additional types of electrolyte sources to provide, for example, magnesium (Mg) ions, can also be included in the electrolyte blend in addition to or independently of sodium (Na). Different types of electrolytes can be provided by their compounds or a combination of their compounds. For example, the magnesium compounds can include magnesium chloride, magnesium carbonate and magnesium sulfate, or a combination thereof.

In one embodiment of a sports beverage, the potassium ions are provided by monopotassium phosphate. In one such embodiment, monopotassium phosphate comprises around about 0.0435% by weight of the beverage composition. In another embodiment, the beverage may contain about 0.01% by weight to about 0.04% by weight of potassium, about 0.01% by weight to about 0.02% by weight of magnesium, about 0.001% by weight to about 0.003% by weight of calcium, about 0.02% by weight to about 0.03% by weight of chloride. Other amounts or combinations may also be useful.

In one embodiment, the potassium ions are provided by monopotassium phosphate or dipotassium phosphate. In one such embodiment, monopotassium phosphate comprises around about 0.0439% by weight of the beverage composition. In another embodiment, the beverage may contain about 0.01% by weight to about 0.04% by weight of potassium, about 0.01% by weight to about 0.02% by weight of magnesium, about 0.001% by weight to about 0.003% by weight of calcium, about 0.02% by weight to about 0.03% by weight of chloride. Other amounts or combinations may also be useful. It is to be understood that any combination of electrolytes from among those listed above and those known to those of skill in the art and envisioned by the present invention.

An edible acid used in the beverages products disclosed herein may serve any one or more of several functions, including, for example, lending tartness to the taste of the beverage, enhancing palatability, increasing thirst quenching effect, modifying sweetness and acting as a mild preservative. Suitable acids are known and will be apparent to those skilled in the art given the benefit of this disclosure. Exemplary acids suitable for use in some or all embodiments of the beverage products disclosed here include phosphoric acid, citric acid, malic acid, tartaric acid, ascorbic acid, lactic acid, formic acid, fumaric acid, gluconic acid, succinic acid, maleic acid, sodium acid sulfate, adipic acid, cinnamic acid, glutaric acid, and mixtures of any of them. Typically, the acid is phosphoric acid, citric acid, malic acid, or combinations thereof such as phosphoric acid and citric acid.

The acid may be used in solution form, for example, and in an amount sufficient to provide the desired pH of the beverage. The particular acid or acids chosen and the amount used will depend, in part, on the other ingredients, the desired shelf life of the beverage product, as well as effects on the beverage pH, titratable acidity, and taste. Typically, for example, the one or more acids of the acidulant are used in an amount, collectively, of from about 0.01% to about 1.0% by weight of the beverage, e.g., from about 0.01% to about 0.5% by weight, from about 0.05% to about 0.5% by weight, from about 0.05% to about 0.25% by weight, from about 0.1% to about 0.25% by weight, depending upon the acidulant used, desired pH, other ingredients used, etc. The pH of at least certain exemplary embodiments of the beverages disclosed here may be a value within the range of from about 2.0 to 5.0, about 2.5 to 4.0, about 2.8 to 3.3 or about 3.0 to 3.2, e.g., 3.1. The acid in certain exemplary embodiments enhances beverage flavor. Too much acid may impair the beverage flavor and result in tartness or other off-taste, while too little acid may make the beverage taste flat.

Those skilled in the art, given the benefit of this disclosure, will recognize that when preparing beverage products containing sweeteners such as peptide-based artificial sweeteners such as aspartame, the resulting beverage composition is best maintained below a certain pH to retain the sweetening effect of the artificial sweetener. In the formation of calcium-supplemented beverages, the presence of calcium salts increases the pH which requires additional acids to both assist the dissolution of the salt and maintain a desirable pH for stability of the artificial sweetener. The presence of the additional acid in the beverage composition, which increases the titratable acidity of the composition, will result in a more tart or sour taste to the resulting beverage. It will be within the ability of those skilled in the art, given the benefit of this disclosure, to select a suitable acid or combination of acids and the amounts of such acids for the acidulant component of any particular embodiment of the beverage products disclosed here.

Sweeteners may be used in the beverage product disclosed herein. Such sweeteners suitable for use in various exemplary embodiments of beverage products include natural and artificial or synthetic sweeteners. Suitable sweeteners and combinations of sweeteners are selected for the desired nutritional characteristics, taste profile for the beverage, mouthfeel and other organoleptic factors. As used herein, “taste” refers to a combination of sweetness perception, temporal effects of sweetness perception, i.e., on-set and duration, off-tastes, e.g. bitterness and metallic taste, residual perception (aftertaste) and tactile perception, e.g. body and thickness. As used herein, a “full-calorie” beverage formulation is one fully sweetened with a nutritive sweetener. The term “nutritive sweetener” refers generally to sweeteners which provide significant caloric content in typical usage amounts, e.g., more than about 5 calories per 8 oz. serving of beverage. As used herein, a “potent sweetener” means a sweetener which is at least twice as sweet as sugar, that is, a sweetener which on a weight basis requires no more than half the weight of sugar to achieve an equivalent sweetness. For example, a potent sweetener may require less than one-half the weight of sugar to achieve an equivalent sweetness in a beverage sweetened to a level of 10 degrees Brix with sugar. Potent sweeteners include both nutritive (e.g., Lo Han Guo juice concentrate) and non-nutritive sweeteners (e.g., typically, Lo Han Guo powder). In addition, potent sweeteners include both natural potent sweeteners and artificial potent sweeteners. However, for natural beverage products disclosed here, only natural potent sweeteners are employed.

Sweeteners suitable for at least certain exemplary embodiments include, for example, sugar alcohols such as sorbitol, mannitol, xylitol, lactitol, isomalt, and malitol. Other sweeteners include tagatose, e.g., D-tagatose, and combinations of tagatose with the sugar alcohol erythritol.

Exemplary natural nutritive sweeteners suitable for some or all embodiments of the beverage products disclosed here include crystalline or liquid sucrose, fructose, glucose, dextrose, maltose, trehalose, fructo-oligosaccharides, glucose-fructose syrup from natural sources such as apple, chicory, honey, etc., e.g., high fructose corn syrup, invert sugar and the like and mixtures of any of them; exemplary artificial sweeteners suitable for some or all embodiments of the beverages disclosed here include saccharin, cyclamate, aspartame, other dipeptides, acesulfame potassium, and other such potent sweeteners, and mixtures of any of them. Also, in at least certain exemplary embodiments of the beverages disclosed here, combinations of one or more natural nutritive sweeteners, one or more artificial sweeteners and/or one or more natural non-nutritive potent sweeteners are used to provide the sweetness and other aspects of desired taste profile and nutritive characteristics. It should also be recognized that certain such sweeteners will, either in addition or instead, act as tastents, masking agents or the like in various embodiments of the beverages disclosed here, e.g., when used in amounts below its (or their) sweetness perception threshold in the beverage in question.

High-potency sweeteners useful in the beverages of the present invention include one or more of a natural high-potency sweetener such as steviol glycosides, such as rebaudiosides such as rebaudioside A, rebaudioside B, rebaudioside C, rebaudioside D, rebaudioside E, rebaudioside F, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobtain, baiyunoside, osladin, polypodoside A, pterocaryoside A, pterocaryoside B, mukurozioside, phlomisoside I, periandrin I, abrusoside A, cyclocarioside I, and combinations thereof.

Sweeteners suitable for use in various embodiments of the beverages disclosed here include nutritive and non-nutritive, natural and artificial or synthetic sweeteners. Non-nutritive artificial sweeteners suitable for at least certain exemplary embodiments include, for example, peptide based sweeteners, e.g., aspartame, neotame, and alitame, and non-peptide based sweeteners, for example, sodium saccharin, calcium saccharin, acesulfame (including but not limited to acesulfame potassium), cyclamate (including but not limited to sodium cyclamate and/or calcium cyclamate), neohesperidin dihydrochalcone, and sucralose. Alitame may be less desirable for caramel-containing beverages where it has been known to form a precipitate. Other non-nutritive sweeteners suitable for at least certain exemplary embodiments include, for example, glycyrrhizin, neohesperidin dihydrochalcone, maltose, lactose, fructo-oligosaccharides, Lo Han Guo powder, steviol glycosides, e.g., rebaudiosides such as Rebaudioside A, stevioside, etc., xylose, arabinose, isomalt, trehalulose, and ribose, and protein sweeteners such as monatin, thaumatin, monellin, brazzein, L-alanine and glycine related compounds and mixtures of any of them. It will be within the ability of those skilled in the art, given the benefit of this disclosure, to select suitable sweeteners or sweetener combinations for a particular embodiment of the beverage compositions disclosed here.

The sweetener can include a monosaccharide or a disaccharide. A certain degree of purity from contamination by metal cations will be expected. Peptides possessing sweet taste are also permitted. The most commonly employed saccharides include sucrose, fructose, dextrose, maltose and lactose and invert sugar. Mixtures of these sugars can be used. Other natural carbohydrates can be used if less or more sweetness is desired. Other types of natural sweeteners structured from carbon, hydrogen and oxygen, e.g., rebaudioside A, stevioside, Lo Han Guo, mogroside V, monatin, can also be used.

Non-limiting examples of nutritive sweeteners include sucrose, liquid sucrose, fructose, liquid fructose, glucose, liquid glucose, glucose-fructose syrup from natural sources such as apple, chicory, agave, honey, etc., e.g., high fructose corn syrup, chicory syrup, Agave syrup, invert sugar, medium invert sugar, maple syrup, maple sugar, honey, brown sugar molasses, e.g., cane molasses and sugar beet molasses, sorghum syrup, an mixtures of any of them.

Sweeteners are present in at least certain exemplary embodiments in an amount of from about 0.1% to about 20% by weight of the beverage, such as from about 6% to about 16% by weight, depending upon the desired level of sweetness for the beverage. To achieve desired beverage uniformity, texture and taste, in certain exemplary embodiments of the natural beverage products disclosed here, standardized liquid sugars as are commonly employed in the beverage industry can be used. Typically such standardized sweeteners are free of traces of non-sugar solids which could adversely affect the flavor, color or consistency of the beverage.

Certain exemplary embodiments of the beverage products disclosed here also may contain small amounts of alkaline agents to adjust pH. Such agents include, e.g., potassium citrate and sodium citrate. For example, the alkaline agent potassium hydroxide may be used in an amount of from about 0.005 wt. % to about 0.02 wt. % (by weight of the beverage), with an amount of about 0.01% being typical for certain beverages. The amount will depend, of course, on the type of alkaline agents and on the degree to which the pH is to be adjusted.

The beverage products disclosed here optionally contain a flavor composition, for example, natural, non-natural and synthetic fruit flavors, botanical flavors, other flavors, and mixtures thereof. As used here, the term “fruit flavor” refers generally to those flavors derived from the edible reproductive part of a seed plant. Included are both those wherein a sweet pulp is associated with the seed, e.g., banana, tomato, cranberry and the like, and those having a small, fleshy berry. The term berry also is used here to include aggregate fruits, i.e., not “true” berries, but fruit commonly accepted as such. Also included within the term “fruit flavor” are synthetically prepared flavors made to simulate fruit flavors derived from natural sources. Examples of suitable fruit or berry sources include whole berries or portions thereof, berry juice, berry juice concentrates, berry purees and blends thereof, dried berry powders, dried berry juice powders, and the like.

Exemplary fruit flavors include the citrus flavors, e.g., orange, lemon, lime grapefruit, tangerine, mandarin orange, tangelo, and pomelo, and such flavors as apple, grape, cherry, and pineapple flavors and the like, and mixtures thereof. In certain exemplary embodiments the beverage concentrates and beverages comprise a fruit flavor component, e.g., a juice concentrate or juice. As used here, the term “botanical flavor” refers to flavors derived from parts of a plant other than the fruit. As such, botanical flavors may include those flavors derived from essential oils and extracts of nuts, bark, roots and leaves. Also included within the term “botanical flavor” are synthetically prepared flavors made to simulate botanical flavors derived from natural sources. Examples of such flavors include cola flavors, tea flavors, and the like, and mixtures thereof. The flavor component may further comprise a blend of several of the above-mentioned flavors. In certain exemplary embodiments of the beverage concentrates and beverages a cola flavor component is used or a tea flavor component. The particular amount of the flavor component useful for imparting flavor characteristics to the beverages of the present invention will depend upon the flavor(s) selected, the flavor impression desired, and the form of the flavor component. Those skilled in the art, given the benefit of this disclosure, will be readily able to determine the amount of any particular flavor component(s) used to achieve the desired flavor impression.

Juices suitable for use in at least certain exemplary embodiments of the beverage products disclosed here include, e.g., fruit, vegetable and berry juices. Juices may be employed in the present invention in the form of a concentrate, puree, single-strength juice, or other suitable forms. The term “juice” as used here includes single-strength fruit, berry, or vegetable juice, as well as concentrates, purees, milks, and other forms. Multiple different fruit, vegetable and/or berry juices may be combined, optionally along with other flavorings, to generate a beverage having the desired flavor. Examples of suitable juice sources include plum, prune, date, currant, fig, grape, raisin, cranberry, pineapple, peach, banana, apple, pear, guava, apricot, Saskatoon berry, blueberry, plains berry, prairie berry, mulberry, elderberry, Barbados cherry (acerola cherry), choke cherry, date, coconut, olive, raspberry, strawberry, huckleberry, loganberry, currant, dewberry, boysenberry, kiwi, cherry, blackberry, quince, buckthorn, passion fruit, sloe, rowan, gooseberry, pomegranate, persimmon, mango, rhubarb, papaya, litchi, lemon, orange, lime, tangerine, mandarin and grapefruit etc. Numerous additional and alternative juices suitable for use in at least certain exemplary embodiments will be apparent to those skilled in the art given the benefit of this disclosure. In the beverages of the present invention employing juice, juice may be used, for example, at a level of at least about 0.2% by weight of the beverage. In certain exemplary embodiments juice is employed at a level of from about 0.2% to about 40% by weight of the beverage. Typically, juice may be used, if at all, in an amount of from about 1% to about 20% by weight.

Certain such juices which are lighter in color may be included in the formulation of certain exemplary embodiments to adjust the flavor and/or increase the juice content of the beverage without darkening the beverage color. Examples of such juices include apple, pear, pineapple, peach, lemon, lime, orange, apricot, grapefruit, tangerine, rhubarb, cassis, quince, passion fruit, papaya, mango, guava, litchi, kiwi, mandarin, coconut, and banana. Deflavored and decolored juices may be employed if desired.

Other flavorings suitable for use in at least certain exemplary embodiments of the beverage products disclosed here include, e.g., spice flavorings, such as cassia, clove, cinnamon, pepper, ginger, vanilla spice flavorings, cardamom, coriander, root beer, sassafras, ginseng, and others. Numerous additional and alternative flavorings suitable for use in at least certain exemplary embodiments will be apparent to those skilled in the art given the benefit of this disclosure. Flavorings may be in the form of an extract, oleoresin, juice concentrate, bottler's base, or other forms known in the art. In at least certain exemplary embodiments, such spice or other flavors complement that of a juice or juice combination.

The one or more flavorings may be used in the form of an emulsion. A flavoring emulsion may be prepared by mixing some or all of the flavorings together, optionally together with other ingredients of the beverage, and an emulsifying agent. The emulsifying agent may be added with or after the flavorings mixed together. In certain exemplary embodiments the emulsifying agent is water-soluble. Exemplary suitable emulsifying agents include gum acacia, modified starch, carboxymethylcellulose, gum tragacanth, gum ghatti and other suitable gums. Additional suitable emulsifying agents will be apparent to those skilled in the art of beverage formulations, given the benefit of this disclosure. The emulsifier in exemplary embodiments comprises greater than about 3% of the mixture of flavorings and emulsifier. In certain exemplary embodiments the emulsifier is from about 5% to about 30% of the mixture.

Carbon dioxide can be used to provide effervescence to certain exemplary embodiments of the beverages disclosed here. Any of the techniques and carbonating equipment known in the art for carbonating beverages may be employed. Carbon dioxide may enhance the beverage taste and appearance and may aid in safeguarding the beverage purity by inhibiting and destroying objectionable bacteria. In certain embodiments, for example, the beverage has a CO₂ level up to about 4.0 volumes carbon dioxide. Typical embodiments may have, for example, from about 0.5 to 5.0 volumes of carbon dioxide. As used here and independent claims, one volume of carbon dioxide is defined as the amount of carbon dioxide absorbed by any given quantity of liquid, e.g., water at 60° F. (16° C.) and one atmospheric pressure. A volume of gas occupies the same space as does the liquid by which it is dissolved. The carbon dioxide content may be selected by those skilled in the art based on the desired level of effervescence and the impact of the carbon dioxide on the taste or mouthfeel of the beverage. The carbonation may be natural or synthetic.

The beverage concentrates and beverages disclosed here may contain additional ingredients beyond the antioxidant compounds of the present invention in amounts sufficient to reduce oxidative modification of cellular proteins and other cellular constituents involved in the process of muscle cell contraction when administered as described herein, including, generally, any of those typically found in beverage formulations. These additional ingredients, for example, may typically be added to a stabilized beverage concentrate. Examples of such additional ingredients include, but are not limited to, caramel and other coloring agents or dyes, antifoaming agents, gums, emulsifiers, tea solids, cloud components, and mineral and non-mineral nutritional supplements.

Examples of non-mineral nutritional supplement ingredients are known to those of ordinary skill in the art and include, for example, vitamins, including Vitamins A, D, E (tocopherol), C (ascorbic acid), B (thiamine), B₂ (riboflavin), B₆, B₁₂, and K, niacin, folic acid, biotin, and combinations thereof. The optional non-mineral nutritional supplements are typically present in amounts generally accepted under good manufacturing practices. Exemplary amounts are between about 1% and about 100% RDV, where such RDV are established. In certain exemplary embodiments the non-mineral nutritional supplement ingredient(s) are present in an amount of from about 5% to about 20% RDV, where established.

Preservatives may be used in at least certain embodiments of the beverages disclosed here. That is, at least certain exemplary embodiments contain an optional dissolved preservative system. Solutions with a pH below 4 and especially those below 3 typically are “microstable,” i.e., they resist growth of microorganisms, and so are suitable for longer term storage prior to consumption without the need for further preservatives. However, an additional preservative system may be used if desired. If a preservative system is used, it may be added to the beverage product at any suitable time during production, e.g., in some cases prior to the addition of the sweetener. As used here, the terms “preservation system” or “preservatives” include all suitable preservatives approved for use in food and beverage compositions, including, without limitation, such known chemical preservatives as benzoates, e.g., sodium, calcium, and potassium benzoate, sorbates, e.g., sodium, calcium, and potassium sorbate, citrates, e.g., sodium citrate and potassium citrate, polyphosphates, e.g., sodium hexametaphosphate (SHMP), and mixtures thereof, and antioxidants such as ascorbic acid, EDTA, BHA, BHT, TBHQ, dehydroacetic acid, dimethyldicarbonate, ethoxyquin, heptylparaben, and combinations thereof. Preservatives may be used in amounts not exceeding mandated maximum levels under applicable laws and regulations. The level of preservative used typically is adjusted according to the planned final product pH, as well as an evaluation of the microbiological spoilage potential of the particular beverage formulation. The maximum level employed typically is about 0.05% by weight of the beverage. It will be within the ability of those skilled in the art, given the benefit of this disclosure, to select a suitable preservative or combination of preservatives for beverages according to this disclosure.

According to methods of the present invention, the antioxidant compound, such as TROLOX, is delivered to individuals in a method to reduce oxidative modification of cellular proteins and other cellular components involved in the process of skeletal muscle cell contraction. The antioxidant compounds are useful in a method of delaying the onset of muscle fatigue and accordingly improving athletic performance by reducing the oxidative modification of cellular proteins and other cellular components involved in the process of muscle cell contraction.

Embodiments of the present invention are also directed to a muscle cell assay that is used to identify compounds useful in reducing oxidative modification of cellular proteins and other cellular components involved in the process of skeletal muscle cell contraction, as well as, to categorize the ability of compounds to be useful in the methods described herein relative to TROLOX. According to this aspect of the present invention, mature myotubes, referred to herein as muscle cells, are exposed to electrical pulse stimulation (EPS) whereupon they repeatedly contract. The muscle cells are stimulated to repeatedly contract for a period of time, such as 90 minutes. The muscle cells are then analyzed to determine the extent of oxidative modification of cell proteins and other cellular components involved in the process of muscle cell contraction. According to one aspect of the present invention, significant oxidative modification of myosin heavy chain protein is created as a result of the repeated muscle cell contraction. Myosin heavy chain protein is involved in the muscle contraction process. According to an additional aspect, myotubes are pretreated with an antioxidant compound at a suitable concentration and for a suitable time period, followed by electrical pulse stimulation and then analyzed to determine the extent of oxidation of myosin heavy chain protein. The extent of oxidation of myosin heavy chain protein in the muscle cells treated with the antioxidant compound is compared to the extent of oxidation of myosin heavy chain protein in the untreated muscle cells. Suitable time periods include between about 30 minutes and about 120 minutes about 60 minutes and 100 minutes, and about 90 minutes. Suitable pulse rates include those which replicate muscle cell contraction during exercise and other pulse rates suitable for generating reactive oxygen species, oxidants and/or free radicals in the muscle cells. Useful pulse frequencies include those between about 0.5 Hz and about 4 Hz, about 1 Hz and about 3 Hz, and any ranges or values in between whether overlapping or not. Useful pulse durations include those between about 2 ms to about 24 ms, about 5 ms to about 20 ms, about 10 ms to about 15 ms and any ranges or value in between whether overlapping or not. Suitable concentrations of a candidate compound range from about 0.001 mM to about 50 mM, about 0.01 mM to about 25 mM, about 0.1 mM to about 10 mM, about 0.5 mM to about 5 mM, about 1 mM and any range or value in between the above ranges whether overlapping or not. A compound that reduces the extent of oxidation of myosin heavy chain protein is identified as an antioxidant compound suitable to reduce muscle fatigue resulting from oxidative modification of cellular proteins and other cellular components involved in the process of muscle contraction. According to certain embodiments, TROLOX is an exemplary antioxidant compound that reduces the oxidation of myosin heavy chain proteins. According to certain embodiments, useful antioxidant compounds reduce oxidative modification by from about 1% to about 99%, from about 10% to about 80%, from about 20% to about 70%, from about 30% to about 60%, from about 40% to about 50% and any range or percentage in between, whether overlapping or not.

The following examples are set forth as being representative of the present invention. These examples are not to be construed as limiting the scope of the invention as these and other equivalent embodiments will be apparent in view of the present disclosure, figures, and accompanying claims.

### Example 1

### Muscle Cell Assay for Determining Oxidation of Cellular Constituents

Mouse C2C12 myoblasts were purchased from American Type Culture Collection (ATCC; Manassas, Va.) and grown to 90% confluence in high glucose Dubecco's Modified Eagle media (DMEM) with 10% fetal bovine serum (FBS). Myoblasts from the purchased lot were grown in T75 flasks and passaged >5 times prior to plating in collagen coated six-well plates for experimentation. To induce myotube differentiation FBS was replaced with 2% horse serum for 5 days. Myoblasts were grown and myotubes were maintained without antibiotics in a 37° C., 5% CO₂ incubator. Western blot analysis for myosin heavy chain (MHC) protein indicated expression by day five with maintenance of steady state protein levels through day eight of differentiation.

Electrical pulsing was applied to the myotubes using a C-Pace EP Cell Culture Stimulator (IonOptix; Milton, Mass.). Cells were stimulated to visibly and repetitively contract for 90 minutes at a pulse frequency of 0.5 Hz and a pulse duration of 24 ms. Immediately prior to stimulation, day old media was removed and replaced with 5 ml of fresh differentiation media.

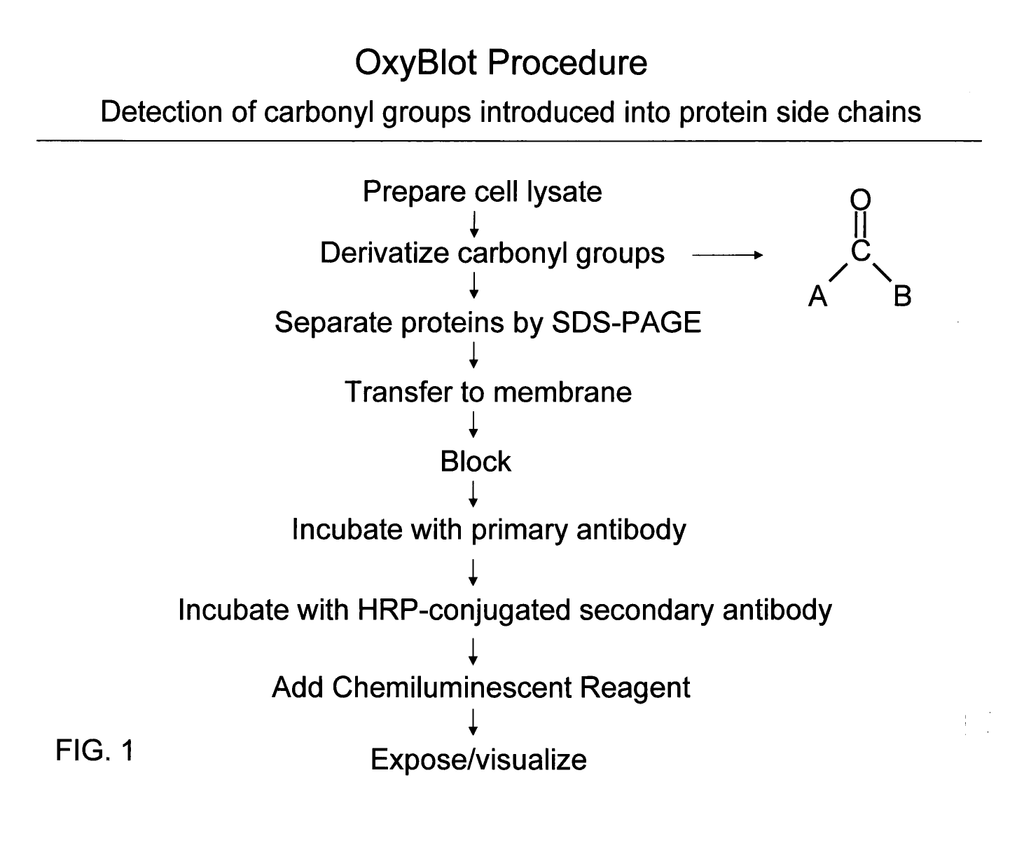

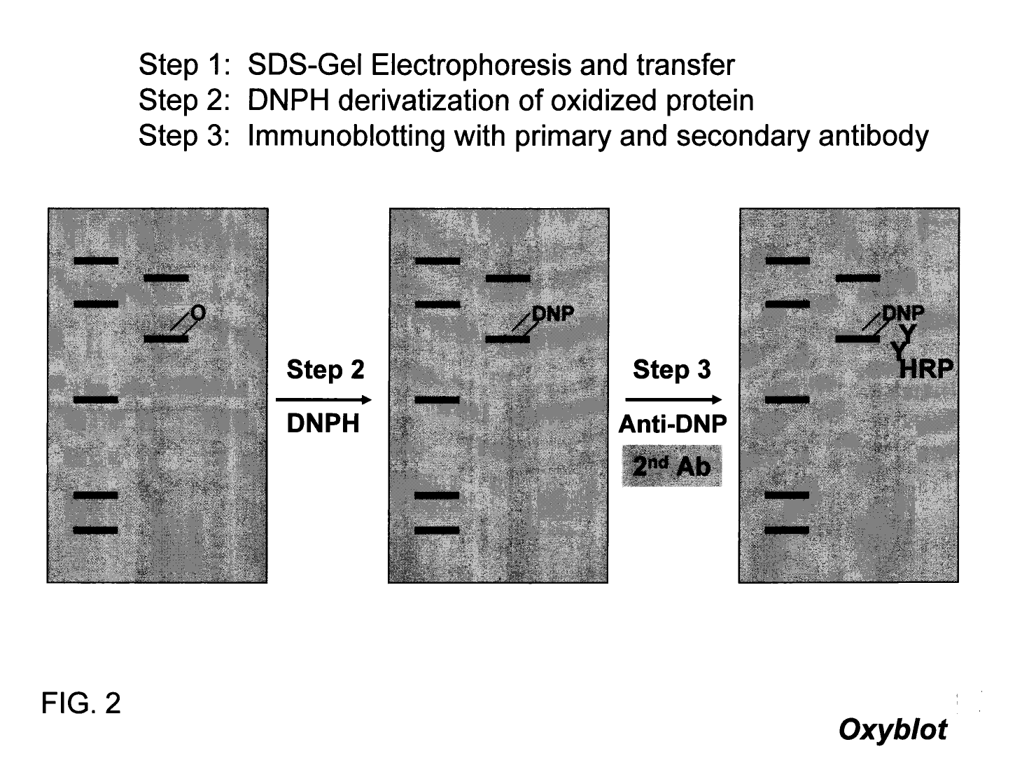

Myotubes were lysed and proteins were derivatized to 2,4-dinitrophenylhydrazone (DNP; OXYBLOT method). Derivatized proteins were separated by gel electrophoresis and transferred to a nitrocellulose membrane for immunoblotting first with a primary antibody directed against DNP proteins and then a HRP-conjugated secondary antibody for visualization. Protein bands were visualized and quantified with a Bio-Rad Chemidoc XRS system. The process for quantifying the amount of oxidized proteins using the OXYBLOT method is shown in FIGS. 1 and 2.

### Example 2

### Muscle Cell Assay with TROLOX

For incubation experiments differentiated myotubes were pre-treated with the water soluble vitamin E analog, TROLOX. Approximately 18 hours prior and again immediately before electrical pulse stimulation, DMEM containing 2% horse serum was replaced with the same media containing TROLOX at a concentration of 1 mM.

### Example 3

### TROLOX Reduced Oxidative Modification of Myosin Heavy Chain Protein

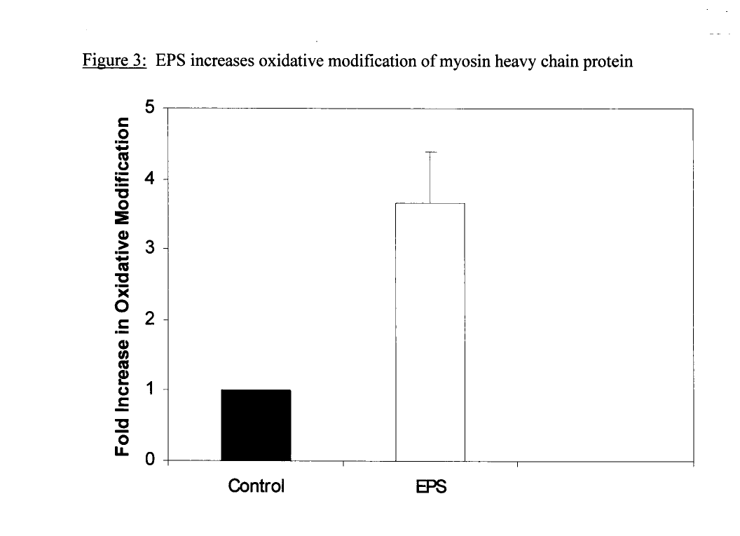

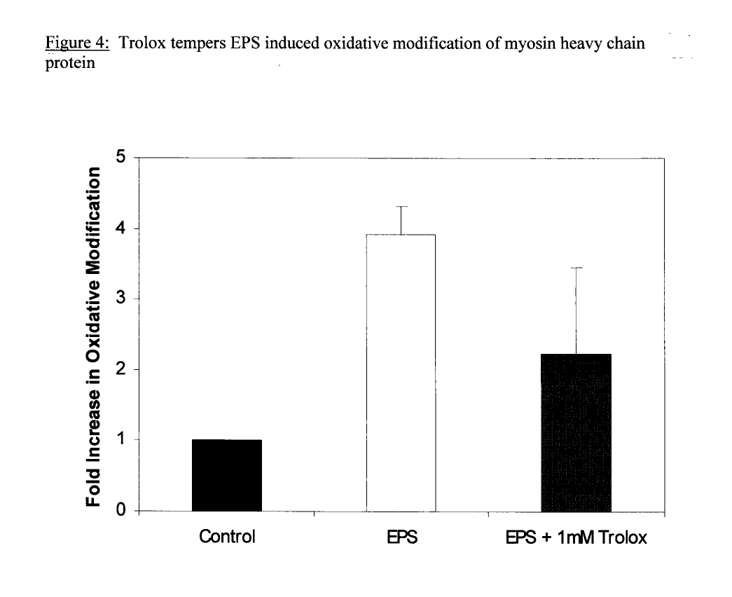

Electrical pulse stimulation of myotubes on day five of differentiation produced visible, repetitive contraction of cells. Relative to unstimulated myotubes, 90 minutes of electrical pulse stimulation at a pulse frequency of 0.5 Hz and duration of 24 ms resulted in a 3.65±0.74 fold increase (p<0.05) in oxidative modification of myosin heavy chain (MHC) protein (six independent experiments) as shown in FIG. 3. Verification of myosin heavy chain protein on OXYBLOT was accomplished by stripping the blot and re-probing with an antibody against myosin heavy chain protein which resulted in a band appearing in the same region relative to the 250 kD molecular weight marker. Oxidative modification of MHC protein was reduced about 42% (42±15% of EPS alone) when cells were pre-treated as in Example 2 with 1 mM TROLOX (five independent experiments) as shown in FIG. 4.

### Example 4

### Screening for Candidate Compounds that Reduce Oxidative Modification of Myosin Heavy Chain Protein

Candidate compounds are screened for their ability to reduce oxidative modification of myosin heavy chain protein. Suitable candidate compounds include the antioxidant compounds describe herein. Differentiated myotubes are pre-treated with the candidate compound, preferably in a water soluble form. Approximately 18 hours prior and again immediately before electrical pulse stimulation, DMEM containing 2% horse serum is replaced with the same media containing the candidate compound at a concentration of between about 0.001 mM to about 10 mM.

Electrical pulse stimulation of differentiated myotubes is carried out to produce visible, repetitive contraction of cells. A pulse frequency of 0.5 Hz and duration of 24 ms for 90 minutes is used. The oxidative modification of myosin heavy chain (MHC) protein is determined and compared to a control experiment lacking the candidate compound or to an experiment where cells are pretreated with a control antioxidant compound such as TROLOX. If the oxidative modification of myosin heavy chain (MHC) protein is reduced in the muscle cells contacted with the candidate compound, then the candidate compound is identified as an antioxidant compound capable of reducing muscle fatigue or improving muscle performance by reducing oxidative modification of cellular proteins and other cellular components involved in the muscle cell contraction process.

### Example 5

### Enhancing Muscle Performance by Reducing Oxidative Modification of Cell Proteins Involved in Muscle Cell Contraction

Muscle performance is analyzed with and without administration of an antioxidant compound using the following protocol. Trained, recreational, healthy, male and female cyclists or triathletes are the test subjects of choice. Body mass, peak oxygen uptake (VO_{2PEAK}), and peak power output, are measured prior to experimental testing. Additionally, a measurement of onset of blood lactate accumulation [OBLA] is made in order to more accurately prescribe the workload, or intensity of exercise, for the performance test measure (i.e., a 2 hour exercise workload representing 95% OBLA followed by a simulated 20-km time trial.) All exercise is performed in a laboratory maintained at 20-25° C. and 35-40% relative humidity with the test subjects exercising on a stationary bicycle trainer calibrated according to manufacturer's recommendations. VO_{2PEAK} is determined using an incremental multistage cycling protocol following a 10-min warm-up at 100 W, athletes cycle at 150 W for five minutes then power output is increased by 50 W every three minutes until 250 W after which power output is increased by 25 W every minute until volitional exhaustion. VO₂ and carbon dioxide production (VCO₂) is computed from expiratory gases collected in Douglas bags. On a second occasion, separated from the measurement of VO_{2PEAK} by at least seven days, test subjects exercise for 3.5 minutes at 55, 60, 65, 70, 75, 80, 85, and 90% VO_{2PEAK}, and 3-mL blood samples are collected during the final 30 sec of each stage for measuring blood lactate concentration and determining OBLA.

Prior to the experimental trials, test subjects perform three familiarization rides, with at least seven days between two trials: i.e., familiarization with the 20-km time trial course, the 2-hour ride followed by the 20-km time trial, and finally the entire testing procedures (2-hour ride followed by the 20-km course in which the subjects are instructed to complete as quickly as possible along with all ancillary measurements and prescribed fluid intakes). Participants then complete experimental exercise trials, in this case one with prior antioxidant treatment, such as TROLOX, and one without in a double blind randomized fashion with at least seven days between the two trials.

For each trial, participants report to the lab at 5 AM following a 10-hour overnight fast, having abstained from exercise over the preceding 24 hours. After voiding and verifying that urine specific gravity is <1.020, body mass is recorded, and an intravenous catheter (BD Insyte Autoguard, Becton, Dickinson Infusion Therapy Systems Inc., Sandy Utah) is inserted into an antecubital vein to collect blood samples throughout the trial. Following a 10-min warm-up at 100 W, participants begin the 2-hour constant load ride. Gas exchanges and heart rate are measured every 15 minutes. At regular intervals during the 2-hour constant load ride, blood samples, are collected for the measurement of plasma metabolites and hormones. Two minutes after completing the 2-hour ride, which allows for the removal of the catheter and heart rate monitor, participants begin the simulated 20-km time trial on an undulating course (9.04 km of incline at a 2% average slope and 10.96 km of decline at an −1.95% average slope: ˜180 m uphill and ˜213 m downhill) and are asked to complete the course as quickly as possible. Participants are aware of their position on the course but no verbal stimuli or other information is given.

During each 2-hour ride, participants ingested 2,000 mL of a beverage including an antioxidant compound, such as TROLOX. An exemplary beverage includes a carbohydrate at a level of 4% (a mixture of fructose, galactose and maltodextrins), electrolytes such as sodium, potassium chloride, calcium and magnesium, natural flavoring, and TROLOX at a 1 mM concentration, i.e. 250 mg/L.

Performance is measured by the time it takes to complete the simulated 20-km course or by the average power output maintained during the 20-km ride. Higher power output indicates improved muscle and/or athletic performance. Decreased time to finish the 20-km ride also indicates improved muscle and/or athletic performance. Meaningful improvements in muscle and/or athletic performance in trained athletes over an experimental control within the scope of the present invention includes above about 0.5%, above about 1.0%, above about 1.5%, above about 2.0% and higher. A certain embodiment includes a meaningful improvement in muscle and/or athletic performance in trained athletes over an experimental control of about 1.1%.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

Given the benefit of the above disclosure and description of exemplary embodiments, it will be apparent to those skilled in the art that numerous alternative and different embodiments are possible in keeping with the general principles of the invention disclosed here. Those skilled in this art will recognize that all such various modifications and alternative embodiments are within the true scope and spirit of the invention. While the invention has been illustrated and described in detail in the drawings and foregoing description, such illustration and description is to be considered as exemplary and not restrictive in character, it being understood that, only the preferred embodiments have been shown and described and that all changes and modifications that come within the spirit of the invention are desired to be protected. The appended claims are intended to cover all such modifications and alternative embodiments. It should be understood that the use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

What is claimed is:

1. A method of reducing oxidative modification of one or more proteins or other cellular constituents involved in muscle cell contraction comprising administering one or more antioxidant compounds to an individual engaged in an exercise regimen in an amount sufficient to reduce oxidative modification of the one or more proteins or other cellular constituents.

2. The method of claim 1 wherein the protein is myosin heavy chain protein.

3. The method of claim 2 wherein the antioxidant compound is TROLOX.

4. A method of reducing muscle fatigue in an individual comprising administering one or more antioxidant compounds to an individual engaged in an exercise regimen in an amount sufficient to reduce oxidative modification of one or more proteins or other cellular constituents involved in muscle cell contraction.

5. The method of claim 4 wherein the protein is myosin heavy chain protein.

6. The method of claim 5 wherein the antioxidant compound is TROLOX.

7. A method of increasing muscle performance during exercise comprising administering one or more antioxidant compounds to an individual engaged in an exercise regimen in an amount sufficient to reduce oxidative modification of one or more proteins or other cellular constituents involved in muscle cell contraction.

8. The method of claim 7 wherein the protein is myosin heavy chain protein.

9. The method of claim 8 wherein the antioxidant compound is TROLOX.

10. A method of identifying a compound effective to reduce oxidative modification of one or more proteins or other cellular constituents involved in muscle cell contraction comprising
subjecting a first set of muscle cells to electrical pulse stimulation,
measuring oxidative modification in the first set of muscle cells of one or more proteins or other cellular constituents involved in muscle cell contraction,
contacting a second set of muscle cells with an antioxidant compound,
subjecting the second set of muscle cells to electrical pulse stimulation,
measuring oxidative modification in the second set of muscle cells of one or more proteins or other cellular constituents involved in muscle cell contraction,
comparing the oxidative modification of the first set of muscle cells with the second set of muscle cells, and
identifying the antioxidant compound as effective to reduce oxidative modification of the protein or other cellular constituent involved in muscle cell contraction when the oxidative modification in the second set of muscle cells is less than the oxidative modification in the first set of muscle cells.

11. The method of claim 10 wherein the protein is myosin heavy chain protein.

12. A method of identifying compounds effective to reduce muscle fatigue or increase muscle performance comprising
subjecting a first set of muscle cells to electrical pulse stimulation,
measuring oxidative modification in the first set of muscle cells of one or more proteins or other cellular constituents involved in muscle cell contraction,
contacting a second set of muscle cells with an antioxidant compound,
subjecting the second set of muscle cells to electrical pulse stimulation,
measuring oxidative modification in the second set of muscle cells of one or more proteins or other cellular constituents involved in muscle cell contraction,
comparing the oxidative modification of the first set of muscle cells with the second set of muscle cells, and
identifying the antioxidant compound as effective to reduce muscle fatigue or increase muscle performance when the oxidative modification in the second set of muscle cells is less than the oxidative modification in the first set of muscle cells.

13. The method of claim 12 wherein the protein is myosin heavy chain protein.

14. A method of identifying effectiveness of a compound relative to a control compound to reduce oxidative modification of a protein or other cellular constituent involved in muscle cell contraction, to reduce muscle fatigue or to increase muscle performance comprising
contacting a first set of muscle cells with a control antioxidant compound,
subjecting the first set of muscle cells to electrical pulse stimulation,
measuring oxidative modification in the first set of muscle cells of one or more proteins or other cellular constituents involved in muscle cell contraction,
contacting a second set of muscle cells with a candidate antioxidant compound,
subjecting the second set of muscle cells to electrical pulse stimulation,
measuring oxidative modification in the second set of muscle cells of one or more proteins or other cellular constituents involved in muscle cell contraction,
comparing the oxidative modification of the first set of muscle cells with the second set of muscle cells, and
identifying the effectiveness of the candidate antioxidant compound to reduce oxidative modification of one or more proteins or other cellular constituents involved in muscle cell contraction, to reduce muscle fatigue or to increase muscle performance as more or less effective than the control antioxidant compound based on whether the oxidative modification of the second set of muscle cells is higher or lower than the oxidative modification of the first set of muscle cells.

15. The method of claim 14 wherein the protein is myosin heavy chain protein.

16. A beverage comprising an antioxidant compound in an amount effective to reduce oxidative modification of one or more proteins or other cellular constituents involved in muscle cell contraction when administered to an individual engaged in an exercise regimen.

17. The beverage of claim 16 wherein the antioxidant compound is TROLOX and the protein is myosin heavy chain protein.

18. A beverage comprising an antioxidant compound in an amount effective to reduce muscle fatigue when administered to an individual engaged in an exercise regimen.

19. The beverage of claim 18 wherein the antioxidant compound is TROLOX and the protein is myosin heavy chain protein.

20. A beverage comprising an antioxidant compound in an amount effective to increase muscle performance when administered to an individual engaged in an exercise regimen.

21. The beverage of claim 20 wherein the antioxidant compound is TROLOX and the protein is myosin heavy chain protein.

22. A kit for analysis of oxidative modification of one or more proteins or other cellular components of muscle cell contraction comprising differentiated myotubes, TROLOX, and one or more antioxidant compounds.
